(19) United States
(12) Patent Application Publication
(10) Pub. No.: US 2011/0021653 A1
(43) Pub. Date: Jan. 27, 2011

(54) HYDROGEL COMPATIBLE TWO-PHOTON INITIATION SYSTEM

(76) Inventors: Lixin Zheng, Kirkland, WA (US); Cody A. Young, Phoenix, AZ (US)

Correspondence Address: GEORGE A LEONE, SR;CITADEL PATENT LAW, 9124 Gravelly Lake Drive SW, SUITE 102, Lakewood, WA, 98499, US

(21) Appl. No.: 12/507,723

(22) Filed: Jul. 22, 2009

(51) Int. Cl. C08J 3/28; A61L 27/14; C07C 211/55

(52) U.S. Cl. 522/2; 523/114; 564/427

(57) ABSTRACT

A method for creating 3D structures using two-photon direct writing includes mixing a resin having a monomer structure with a solvent to create a resin-solvent mixture. A chromophore initiator is added to the resin-solvent mixture to create a second mixture, where the chromophore initiator is comprised of a first constitutional unit derived from a compound having the formula

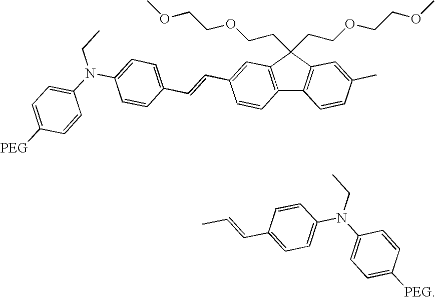

Two-photon lithography is applied to the second mixture to produce a 3D structure.

### TECHNICAL FIELD

The present invention relates to two-photon initiation systems in general, and, more particularly, to a two-photon initiation system using chromophores to initiate two-photon polymerization of hydrogels for 3D construction of structures for promoting cell growth.

### BACKGROUND

Two-photon absorption has been long known as the simultaneous absorption of two photons of identical or different frequencies in order to excite a molecule from one state (usually the ground state) to a higher energy electronic state via a virtual state transition. Two-photon absorption may be used to precisely fabricate three dimensional hydrogel structures in a process called two-photon lithography (TPL). Hydrogel structures have gained importance because they are useful for a number of biomedical applications such as construction of biosensors, tailoring materials for drug delivery, and creating biocompatible scaffolds that interact with living cells.

Key aspects to consider in making patterned structures include the resolution of TPL, the ease of the technique and the freedom to make arbitrary structures. TPL holds promise for accomplishing these key aspects. For example, TPL-fabricated structures include cantilevers and gratings and for creating defects in photonic crystals. Structures can be fabricated from commercially available monomers, of almost any shape with resolution less than 100 nm. However, while TPL has been used to fabricate a wide variety of arbitrarily shaped structures, it has limitations due to the lack of efficient initiators compatible with hydrogels.

One of the major limitations of TPL used in hydrogel fabrication has been the inability to use effective hydrophobic chromophores in aqueous media. This characteristic has limited the use of TPL for biological applications. Unfortunately, the use of cytotoxic organic solvents such as toluene to solubilize the hydrophobic chromophores is very undesirable because changes from organic solvents used in fabrication to then immersing the resultant structure into an aqueous environment can lead to significant structural distortions (See Chemistry of Materials 2009, 21(10), 2003).

As indicated above, known systems are missing a highly efficient two-photon initiator compatible with two-photon initiated hydrogel polymerization. Because currently known initiators are not efficient, high powered and expensive femto-second laser devices must be used to initiate the two-photon polymerization. See, for example, Campagnola, P. J., J. Biomed. Mater. Res., Part A 2004, 71A, 359 and Shear, J. B., Proc. Natl. Acad. Sci. U.S.A. 2004, 101, 16104. Another approach requires the use of a hydrophobic two-photon chromophore and initiator system to form hydrogel structures.

While other 3D fabrication attempts have been tried, they lack the simplicity of a direct fabrication approach such as TPL. Examples of such alternate technologies include pseudo-3D techniques such as using conventional layer-by-layer 2D approaches. A number of varying approaches are reviewed in Peltola, et al., “A review of rapid prototyping techniques for tissue engineering purposes,” Annals of Medicine (2008) Vol. 40: 268-280. Such approaches result in overly complicated fabrication requiring precision alignment and produce structures having interfaces with weak bonding properties. Known approaches are also typically limited in the ability to produce fine features in structures.

As a result there is a need in the art, satisfied for the first time by the methods and compositions disclosed herein, to provide a true 3D fabrication of a hydrogel material with resulting advantages including a simplified fabrication process and improved bonding properties. As a further advantage, an improved TPL as disclosed hereinbelow provides high resolution in creating structures having features of submicron to nanometer resolution over conventional approaches having features of micron to tens of micron resolution. As a result, fine 3D structures may be fabricated, such as, for example, hydrogel cell scaffolding. Further, high efficiency of novel two-photon initiators disclosed for the first time hereinbelow allow fabrication at faster speeds and/or use of a low peak power laser.

### BRIEF SUMMARY OF THE DISCLOSURE

A compound or composition is disclosed which includes:

at least one chromophore initiator compatible with a hydrogel oligomer or polymer, wherein the chromophore has a simultaneous two-photon absorptivity; and

at least one monomer in close proximity to said chromophore, wherein the at least one monomer includes a hydrogel oligomer or polymer.

In one embodiment the chromophore initiator includes a constitutional unit derived from the formula

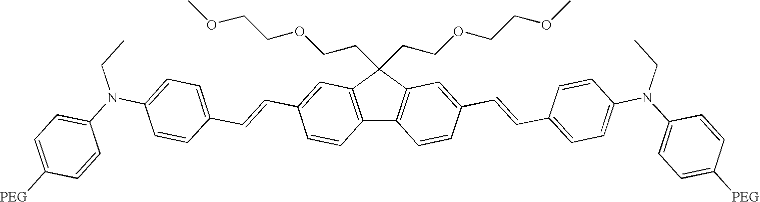

wherein PEG consists of polyethylene glycol.

In another embodiment a method for creating 3D structures using two-photon direct writing is disclosed. The method includes mixing a resin having a monomer structure with a solvent to create a resin-solvent mixture. A chromophore initiator is added to the resin-solvent mixture to create a second mixture, where the chromophore initiator is comprised of a first constitutional unit derived from a compound having the formula

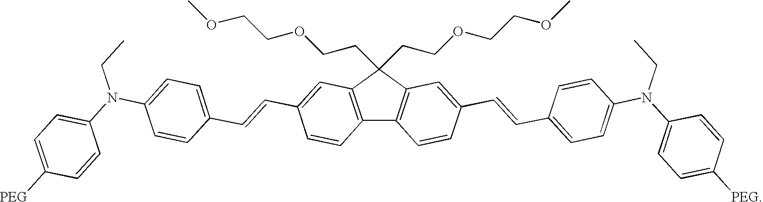

Two-photon lithography is applied to the second mixture to produce a 3D structure.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawings, in which:

FIG. 1 shows a prophetic example of the synthesis of a constitutional unit including a chromophore.

FIG. 2 shows an example of a cross-section of an exemplified 3D scaffold hydrogel structure fabricated by cross-hatched raster scanned two-photon polymerization.

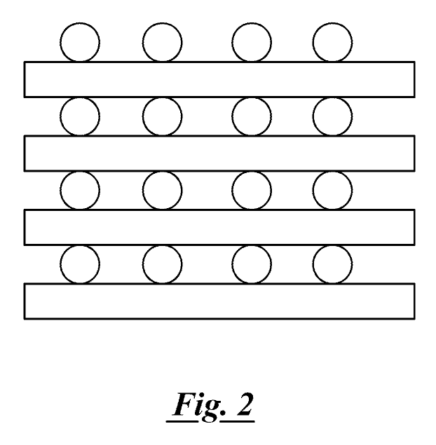

FIG. 3 schematically shows an example of a two-photon polymerization system confocal fiber imaging scheme to irradiate chromophores to initiate two-photon polymerization of hydrogels for 3D construction and imaging of structures for promoting cell growth.

FIG. 4 schematically shows a design for a process flow of one example of a two-photon lithography system.

In the drawings, identical reference numbers identify similar elements or components. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

### DETAILED DESCRIPTION OF THE PREFERRED EMBODIMENTS

The following disclosure describes several embodiments and systems for a two-photon polymerization system using chromophores to initiate two-photon polymerization of hydrogels for 3D construction of structures for promoting cell growth. Several features of methods and systems in accordance with example embodiments are set forth and described in the Figures. It will be appreciated that methods and systems in accordance with other example embodiments can include additional procedures or features different than those shown in the Figures. Example embodiments are described herein with respect to biological cells. However, it will be understood that these examples are for the purpose of illustrating the principles, and that the invention is not so limited.

Additionally, methods and systems in accordance with several example embodiments may not include all of the features shown in these Figures. Throughout the Figures, like reference numbers refer to similar or identical components or procedures.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one example” or “an example embodiment,” “one embodiment,” “an embodiment” or various combinations of these terms means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In one example embodiment this disclosure provides a method of creating 3D cell scaffolds using two-photon direct writing using highly efficient two-photon initiators that are compatible with hydrogel medium. Using the methods and compositions taught herein solves problems present in other techniques while allowing the use of low-cost femtosecond fiber laser, made possible due to the high initiation efficiency of the new initiators.

### 1. Two-Photon Chromophores Compatible With Hydrogels

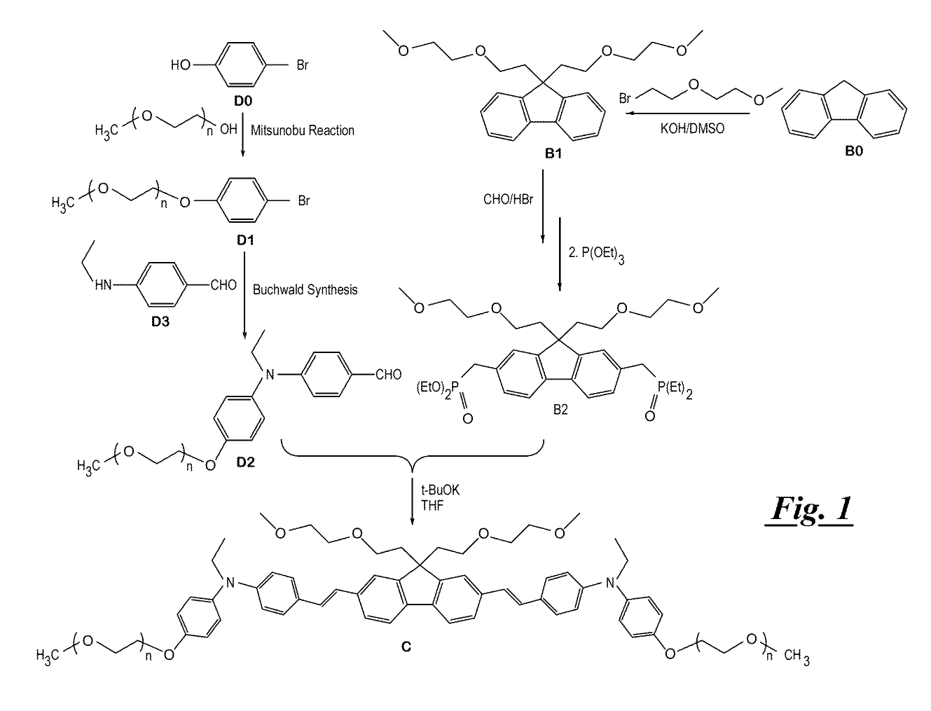

Referring now to FIG. 1 a prophetic example of the synthesis of a constitutional unit including a chromophore, herein also called a chromomer C, is schematically shown. Presented herein below are new two-photon chromophores compatible with hydrogels.

### Prophetic Example 1

### Synthesis of B1:

To a solution of fluorine (10 g, 60 mmol), 1-bromo-2-(2-methoxyethoxy)ethane (24.2 g, 132 mmol), tetrabutylammonium bromide (2.5 g) in degassed DMSO (200 mL), is added NaOH aq (50 wt %, 50 mL). After mechanically stirring at room temperature for overnight, the solution is quenched with water (100 mL), and solvents are distilled under vacuum. The residue solid is dissolved in CH₂Cl₂, and washed with HCl aq. (1 M, 100 mL×3). The organic layer is dried over MgSO₄, and purified by column chromatography to give a viscous liquid.

### Prophetic Example 2

### Synthesis of B2:

A two-necked flask containing a mixture of M1 (10.0 g, 25.9 mmol) and paraformaldehyde (7.68 g, 128 mmol) is placed in an ice bath. A 30% HBr solution (35 mL) in acetic acid is then added carefully to this flask, and the mixture is heated to 70° C. and stirred for 24 h under nitrogen until the HBr is consumed. The hot reaction mixture is cooled to room temperature before it is poured into 200 mL of cold water. The resulting mixture is then extracted with methylene chloride, and the combined organic layer is collected and washed with brine. After drying over anhydrous MgSO₄, evaporation in vacuo afforded the crude product, which is subjected to purification by column chromatography on silica yield a slight yellow transparent liquid. The yield liquid above is heated in excess triethylphosphite (3 eqv.) at 125° C. for 4 hr, and then the excess triethylphosphite is distilled off to M2.

### Prophetic Example 3

### Synthesis of D1:

A flask is charged with bromophenol (1.16 g, 9.6 mmol), methoxypolyethylene glycol (Mn:2000) (19.2 g, 9.6 mmol), triphenylphosphine (PPh₃) (6.1 g, 9.6 mmol), and 150 mL of tetrahydrofuran. The flask is immersed in an ice bath, and diethyl azodicarboxylate (1.6 mL, 9.8 mmol) is added dropwise at a rate such that the temperature of the reaction mixture is maintained below 10° C. Upon completion of the addition, the flask is removed from the ice bath and the solution is allowed to stir at room temperature overnight and subsequently at 40° C. for 3 hr. The reaction mixture is cooled to room temperature, diluted with 150 mL of ether, and washed twice with 100 mL portions of saturated aqueous sodium bicarbonate solution. The aqueous layers are combined and back-extracted with 100 mL of ether. The combined organic layers are dried over sodium sulfate. Excess solvent and other volatile reaction components are completely removed under reduced pressure initially on a rotary evaporator and then under high vacuum. The resulting solid is purified under a flash chromatography column.

### Prophetic Example 4

### Synthesis of D2:

To a solution of tris(dibenzylideneacetone)dipalladium [Pd₂(dba)₃] (0.0274 g, 0.15 mol % relative to the aniline) and bis(diphenylphosphino)ferrocene (DPPF) (0.025 g, 0.225 mol % relative to the aniline) in dry THF (50 mL) under nitrogen is added D1 (4.31 g, 2 mmol) at room temperature, and the resultant mixture is stirred at that temperature for 10 min. Sodiumtert-butoxide (0.25 g, 2.6 mmol) and D3 (0.298 g, 2 mmol) are added to this solution and stirred at 90° C. for 4 h. The reaction mixture is cooled to room temperature and poured into water. The mixture is extracted by toluene (3×100 mL), and the fractions of organic layers are collected together and concentrated in vacuo to give the crude reaction mixture. Purification of the reaction mixture is done by flash column chromatography.

### Prophetic Example 5

### Synthesis of Chromomer (C)

To a stirred solution of D2 (4.61 g, 2 mmol) and B2 (0.639 g, 1 mmol) in THF, is added dropwise a solution of t-BuOK (1 mL, 1M in methanol) at 0° C. After stirring for 2 hr at this temperature, the mixture is quenched with water. THF is removed under reduced pressure, and then the solid is dissolved in methylene chloride, washed with water and dried over Na₂SO₄. Excess solvent is also removed under reduced pressure, and the residue is purified by column chromatography to give the chromomer (C).

One embodiment of the chromomer includes chromophore initiators comprised of a first constitutional unit derived from a compound having the formula

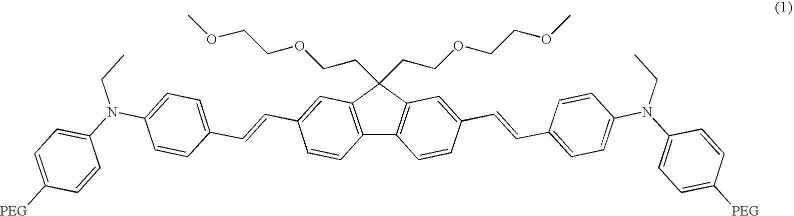

where PEG represents polyethylene glycol.

For the purposes of two-photon polymerization, the chromophore derived from formula (1) is combinable with a resin compound. In one embodiment the resin comprises a compound having the general structure:

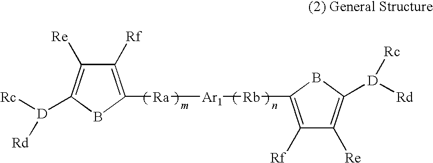

### Where:

D is selected from the group consisting of N, O, S,
m and n are independently selected from the range of integers greater than or equal to zero and less than or equal to six,
B is selected from group consisting CR1=CR2, O, S and N—R3 where R1, R2 and R3 are defined below:
   (i) —H,
   (ii) a linear or branched alkyl group with up to 10 carbons,
   (iii) —(CH2CH2O)x-R, where x: (1-10), R is selected from the group consisting of H and linear branched alkyl group up to 10 carbons or an aryl group, and
   (iv) An aryl group.
   Ra and Rb are selected from the group consisting of the following formulas:

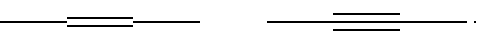

### Rc and Rd:

At least one of Rc and Rd consists of a hydrogel structure (oligomer or polymer), including collagen, collagen-GAG(alginate) copolymers, albumin, hyaluronic acid, fibrinogen-fibrin, chitosan, matrigel, alginate, polyhydroxyalkanoates, starch, poly(lactic acid), poly(d-lactic acid), poly(l-lactic acid), poly(d,l-lactic acid), poly(glycolic acid), poly(lactic-co-glycolic acid), poly(e-caprolactone), poly(hydroxyalkanoate), poly(3 or 4-hydroxybutyrate), poly(3-hydroxyoctanoate), poly(3-hydroxyvalerate), poly (p-dioxanone), poly(propylene fumarate), poly (1,3-trimethylene carbonate), poly(glycerol-sebacate), poly(ester urethane), polyethylene glycol and hydroxyethyl methacrylate (HEMA) and combinations thereof.

One of Rc and Rd is not present when B is O or S. Note that Matrigel™ Basement Membrane Matrix is manufactured by BD BioSciences, NJ and according to their literature is a solubilized basement membrane preparation extracted from the Engelbreth-Holm-Swarm (EHS) mouse sarcoma, a tumor rich in ECM proteins.
Ar₁ is selected from the group consisting of:

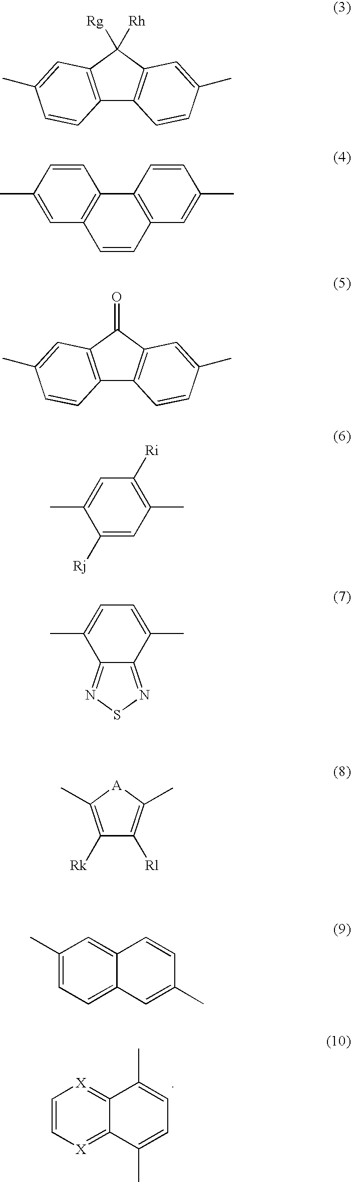

Re,Rf,Rg, Rh, Ri, Rj, Rk, and Rl are selected from the group consisting of:
   (i) —OR4, —CN, —SR5, —F, —Cl, —Br, —I, —NO2, —H,
   (ii) A linear or branched alkyl group with up to 10 carbons,
   (iii) —(CH2CH2O)x-R, where x: (1-10), R is selected from the group consisting of H and linear branched alkyl group up to 10 carbons, or
   (iv) An aryl group selected from the group consisting of phenyl, naphthyl, furanyl, thiophenyl, pyrrolyl, selenophenyl.
      In the example, X is selected from C and N.

### 2. The Chromophores Enable Two-Photon Polymerization of the Hydrogels

As shown in the examples below, a chromophore according to formula (1) provides highly efficient photoinitiation for a two-photon polymerization process.

### 3. Applications:

### Prophetic Example 6

### With Co-Photoinitiator

A typical resin having a monomer structure as shown below in formula (11) is mixed with solvent. An initiator, Irgacure® 651, is added at 0.5% (w/w), based on the monomers. A chromophore according to formula (1) is added having a concentration in a range of 0.5-5 wt %, based on the monomers. All the components are stirred using a stir bar for about 2 hours to form a hydrogel medium. The hydrogel medium is then ready for two-photon lithography.

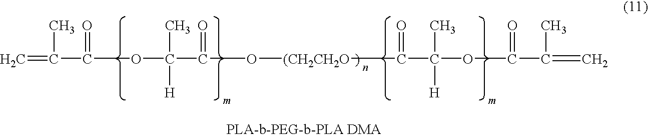

As published by the manufacturer, Ciba Specialty Chemicals, Inc., Basel, Switzerland, Irgacure® 651 is a general-purpose, highly efficient solid, free-radical photoinitiator used for the curing of unsaturated pre-polymers in the chemical class of Benzyldimethyl-ketal having a chemical formula Alpha, alpha-dimethoxy-alpha-phenylacetophenone. It will be apparent to those skilled in the art having the benefit of this disclosure that the co-photoinitiator is not limited to Irgacure® 651, but other equivalent photoinitiators may be used such as, for example, Irgacure® 819 and known equivalents.

### Prophetic Example 7

### Without Co-Photoinitiator

A typical resin having a monomer structure as shown above is mixed with solvent. A chromophore according to formula (1) is added having a concentration in a range of 0.5-5 wt % based on the monomers. All the components are stirred using a stir bar for about 2 hours to form a hydrolgel medium. The hydrogel medium is then used for two-photon lithography.

### 4. Two-Photon Polymerization System

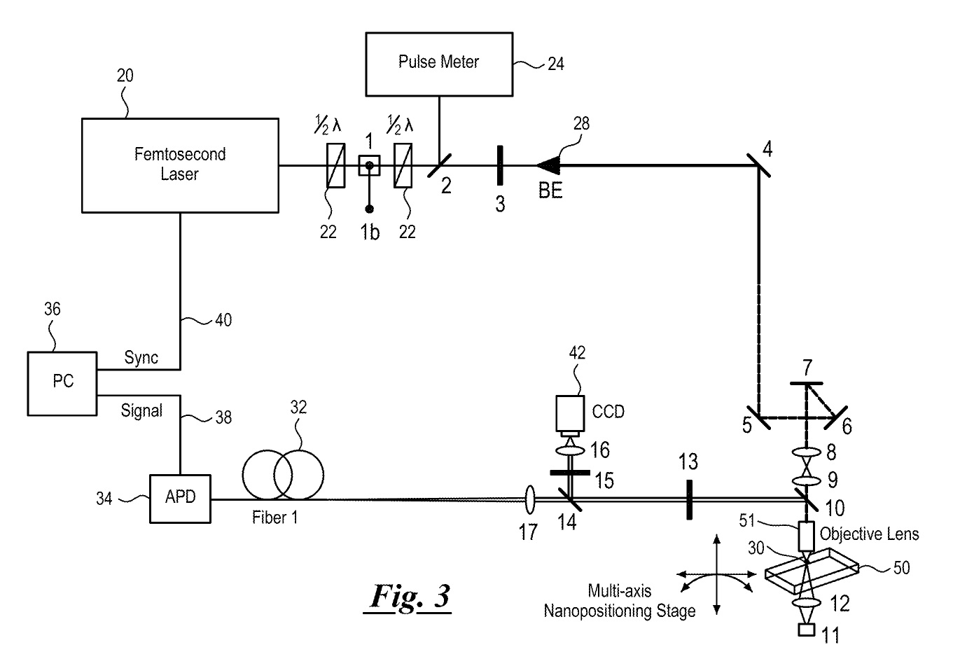

Referring now to FIG. 3, an example of a two-photon polymerization system design using a fiber laser to irradiate chromophores to initiate two-photon polymerization of hydrogels for 3D construction of structures for promoting cell growth is schematically shown. A two-photon lithography system will include a femtosecond laser 20; a first half-wave plate for rotation 22, a polarizing beam splitter 1, a beam dump 1b, a flip mirror 2 and a pulse meter 24 coupled to the femtosecond laser 20 through the flip mirror 2. In operation, a cluster including the first and second half-wave plates, the polarizing beam splitter 1, and the beam dump 1b will be combined to form a polarization controllable variable attenuator. The entire cluster is optional depending on the primary femtosecond laser specifications.

Also located in the beam path will be a computer controlled high speed shutter 3 (for example a shutter, beam slicer or pulse picker); a beam expander 28, a turning (ultrafast) mirror 4, an (optional) turning (ultrafast) mirror 5, (optional) computer controlled steering (ultrafast) mirrors 6,7, steering lens 8, a tube lens 9, a laser line dichroic mirror 10, an objective lens 51, an LED 11, a focuser (dark field and bright field) 12, a fluorescence spot 30, an (optional) polarizer for anisotropy measurement, an (optional) dichroic mirror 14; an IR filter 15, a fiber collector lens 17 and an (optional) multimode confocalization fiber 32. A Personal Computer (PC) 36 will operate to send a control signal to an Avalanche Photodiode (APD) 34 and a synchronization signal to the femtosecond laser 20. A multi-axis nanopositioning stage 50 is adapted to holds and manipulate a substrate for fabrication by two photon polymerization. A CCD camera 42, or equivalent imaging device will be optically located to receive images from the stage.

The laser 20 may include, for example, a femtosecond fiber laser, a Ti:Sapphire femtosecond laser, other conventional ultrafast lasers and equivalents. Those skilled in the art having the benefit of this disclosure will recognize that various other configurations may also be used to accomplish two-photon lithography using the novel principles disclosed herein. Such alternatives may use a dual laser configuration, a de-scanned laser configuration, an inverted beam-path, a commercial microscope system, may have no need for imaging, and may employ only one of either stage-scanned or beam-scanned options. The two-photon cross-section for the chromophore is expected to be ca. 1000 GM. The operation wavelength is expected to be about 780 nm which is a typical fiber laser fixed wavelength. The fabrication speed, NA of the lens, laser peak power and frequency are all related. At 100 um/s fabrication speed, the average laser power (not peak power) of about 5 mW is required with a NA for the lens of about 1.4. Commercially available fiber lasers having parameters of about 100 fs pulse and 100 MHz frequency can provide a laser power up to 65 mW.

Having described one example of a design for two-photon fabrication, it is believed that a discussion of example operational modes will enhance the understanding of this disclosure. Typically, two-photon manufacturing is accomplished when an ultrafast pulsed laser is focused into controlled locations in a reactive monomer (herein referred to as the “material”) to induce a photo-crosslinking polymerization reaction. The wavelength of the laser needs to be such that the material will exhibit very little single-photon absorption and a high amount of two-photon absorption. This is necessary to ensure that a photo-crosslinking reaction will occur tightly confined to the focal spot, deep within the material and not just at the first surface of the material. A shutter controls when the material is exposed to the laser and either a 3D stage, a 2D scanned mirror system (a scan head), beam shaping optics or any combination therein, can be used to control where the material is exposed to the laser light. For ease of language, the 3D stage or scan head are herein referred to as the “intensity distributor”.

The pulse duration, repetition rate, average laser power, numerical aperture of the focusing lens, scan speed of the focal spot in the monomer, two-photon cross section of the absorber, polymerization efficiency, intensity distributor resolution and repeatability, and system losses are all relevant parameters to the manufacturing process. Sufficient enough peak intensity (peak power/area) must be present to induce the threshold limited two-photon photo-crosslinking reaction. With the advent of our super efficient (high two-photon cross section and high polymerization efficiency) materials, as disclosed herein for example, it is now possible to induce the photo-polymerization reaction with greatly reduced peak intensities (less average power, less tightly focusing optics, and longer pulse durations) and thus induce photo-crosslinking over larger areas and in less time than ever before.

The generalized processes by which two-photon fabrication is accomplished is described below with reference to FIG. 3 when pertinent.
1. Ultrafast pulses of long wavelength radiation are produced by the laser 20. As noted above, numerous laser technologies are available which can produce the sufficient peak power (short enough pulse duration with high enough average power over small enough beam diameter) coherent light source.
2. The laser repetition rate is typically reduced from the intrinsic rate of the laser. The repetition period is reduced to below the thermal relaxation time of the material to avoid thermal heating.
3. The laser power is reduced because the intrinsic power of the source is typically too high for precision polymerization. This may be accomplished with two-half wave plates and a polarizing beam splitter to allow fine control over the laser power and polarization angle.
4. The beam is passed through a computer shutter (or with certain laser technologies the laser itself can be gated on and off).
5. The beam is expanded to slightly overfill the back aperture of the objective lens to achieve a tight focus (maximum energy in the smallest possible volume). Expansion of the beam should be accomplished after the shutter to ensure the fastest possible gating of the laser by a mechanical shutter mechanism.
6. The beam may be scanned by a series of computer controlled mirrors. Properly, the laser scan head scans the beam onto a scan lens which is focused onto a back focal plane of the objective lens 51. For infinity corrected objectives (which have no back focal plane), a back focal plane may be achieved with a tube lens.
7. The laser is focused into the material by an objective lens.
8. Laser energy excites the material by two-photons into an excited state. The material can then photo-polymerize directly (depending on the two-photon absorber and monomer system) or the material can fluoresce, thus generating emission photons which are capable of being absorbed by an additional single-photon absorption initiator.
9. The computer controlled stage 50 is translated relative to the focal spot 30. The laser shutter 3 is set to open when it is desired to generate polymer. The simplest method of manufacturing would be to translate the stage 50 to a desired location and then open and close the shutter thus generating a small volume of polymer (a voxel or volume element pixel). This process is repeated at every desired point in the solid until an entire structure is realized. Another simple method is to translate to a first desired point, open the shutter, translate to a second desired point according to a defined path and speed profile, then close the shutter. This generates a line of polymer. This second method is capable of generating a solid built by a series of lines.
10. The photo-crosslinking process just described generates fluorescence. The fluorescence is imaged by the same objective lens which focuses the laser energy, only now with the light traveling back up through the objective. The sample may also be illuminated from behind for dark field or bright field transmission illumination. The fluorescence and illumination light is then be imaged onto a camera (a CCD in the diagram) so as to see the excitation spot.
11. A photo-diode may also be used to image the fluorescence (an APD, such as avalanche photo-diode 34 as shown in FIG. 3). It is possible to reconstruct a full image of the manufactured solid from the photo-diode response once it is synchronized with the known focal position. This technique also enables the manufacturing instrument to be used as a laser scanning microscope.

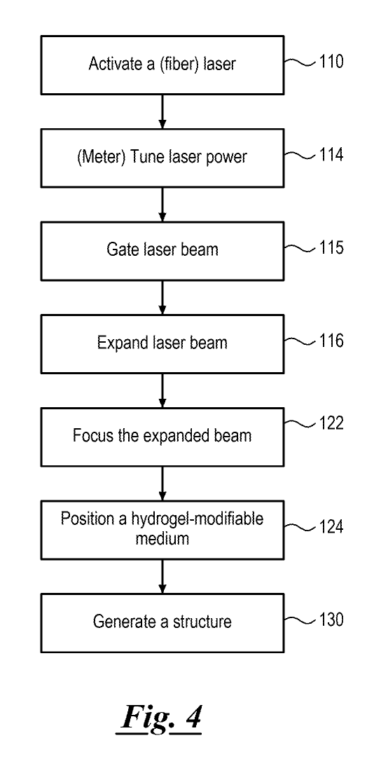

Referring now to FIG. 4, a process flow of one example of operation of a two-photon lithography system is schematically shown. A laser is activated 110 to generate a laser beam along the laser path. Laser beam power is metered 114. The expanded beam is gated 115, as with a shutter in the laser path. The laser beam is 116 with a beam expander coupled to receive the laser beam from the fiber laser to produce an expanded beam. The expanded beam is focused onto a target region using a focusing lens located on the laser path. A hydrogel medium is positioned 124 into the target region along a laser path wherein the hydrogel medium includes a monomer and at least one chromophore having a simultaneous two-photon or two-photon absorptivity, wherein, in one example, the chromophore is derived from a compound having the formula

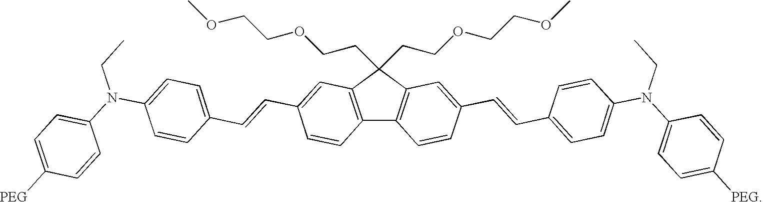

A structure is generated 130 by moving the sample in three dimensions relative to the focusing lens to irradiate said chromophore to cause a simultaneous two-photon or two-photon absorption in said chromophore to produce two photon polymerization of the hydrogel medium. In example embodiments the hydrogel medium is composed of compounds as described hereinabove.

The invention has been described herein in considerable detail in order to comply with the Patent Statutes and to provide those skilled in the art with the information needed to apply the novel principles of the present invention, and to construct and use such exemplary and specialized components as are required. However, it is to be understood that the invention may be carried out by specifically different equipment, and devices, and that various modifications, both as to the equipment details and operating procedures, may be accomplished without departing from the true spirit and scope of the present invention.

What is claimed is:

1. A chromophore initiator comprising a constitutional unit derived from the formula 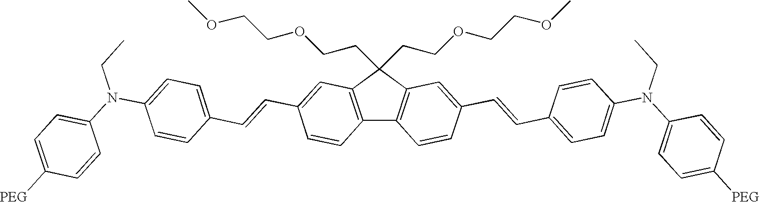
wherein PEG consists of polyethylene glycol.

2. A method for creating 3D structures using two-photon polymerization, the method comprising:
mixing a resin having a monomer structure with a solvent to create a resin-solvent mixture;
adding a chromophore initiator to the resin-solvent mixture to create a second mixture, wherein the chromophore initiator is comprised of a first constitutional unit derived from a compound having the formula 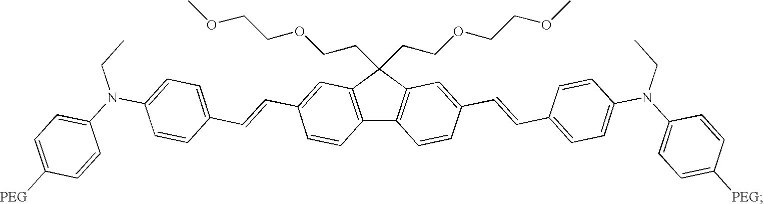
and
applying two-photon lithography to the second mixture to produce a 3D structure.

3. The method of claim 2 wherein the chromophore concentration is in a range of 0.5-5 wt % based on the monomer structure.

4. The method of claim 2 wherein the 3D structure comprises a 3D cell scaffold.

5. The method of claim 2 wherein the resin comprises a compound having the formula 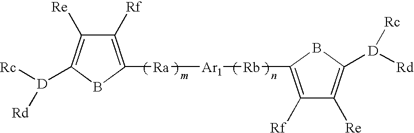
wherein D is selected from the group consisting of N, O, S;
wherein m and n are independently selected from the range of integers greater than or equal to zero and less than or equal to six;
wherein B is selected from group consisting CR1=CR2, O, S and N—R3, wherein R1, R2 and R3 are selected from the group consisting of
(i) —H,
(ii) A linear or branched alkyl group with up to 10 carbons,
(iii) —(CH2CH2O)x-R, where x: (1-10),
R is selected from the group consisting of H and linear branched alkyl group up to 10 carbons or an aryl group, and
(iv) An aryl group;
wherein Ra and Rb are selected from the group consisting of the following formulas: 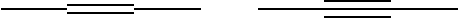
wherein at least one of Rc and Rd consists of a hydrogel structure;
wherein, one of Rc and Rd is not present when B is O or S;
wherein Ar₁ has a formulation selected from the group consisting of 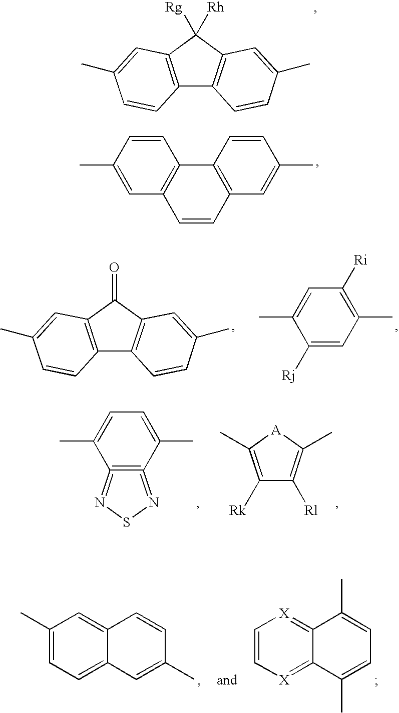
wherein Re, Rf, Rg, Rh, Ri, Rj, Rk, and Rl are selected from the group consisting of
(i) —OR4, —CN, —SR5, —F, —Cl, —Br, —I, —NO2, —H,
(ii) a linear or branched alkyl group with up to 10 carbon,
(iii) —(CH2CH2O)x-R, where x: (1-10), R is selected from the group consisting of H and linear branched alkyl group up to 10 carbons,
(iv) an aryl group selected from the group consisting of phenyl, naphthyl, furanyl, thiophenyl, pyrrolyl, selenophenyl; and
wherein X is selected from the group consisting of C and N.

6. The method of claim 2 wherein the hydrolgel structure is selected from the group consisting of oligomer or polymer, collagen, collagen-GAG(alginate) copolymers, albumin, hyaluronic acid, fibrinogen-fibrin, chitosan, matrigel, alginate, polyhydroxyalkanoates, starch, poly(lactic acid), poly(d-lactic acid), poly(l-lactic acid), poly(d,l-lactic acid), poly(glycolic acid), poly(lactic-co-glycolic acid), poly(e-caprolactone), poly(hydroxyalkanoate), poly(3 or 4-hydroxybutyrate), poly(3-hydroxyoctanoate), poly(3-hydroxyvalerate), poly (p-dioxanone), poly(propylene fumarate), poly (1,3-trimethylene carbonate), poly(glycerol-sebacate), poly(ester urethane), polyethylene glycol, hydroxyethyl methacrylate (HEMA) and combinations thereof.

7. The method of claim 2 further comprising adding a co-photoinitiator to the resin-solvent mixture.

8. The method of claim 8 wherein the co-photoinitiator comprises a free-radical photoinitiator added at 0.5% (w/w) based on the monomers.

9. The method of claim 2 wherein the monomer structure is derived from the formula

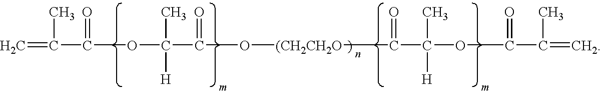

10. The method of claim 3 wherein the monomer structure is derived from the formula

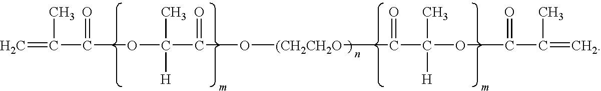

11. A method for fabrication of three-dimensional (3D) hydrogel structures using two-photon polymerization, the method comprising:
activating a laser to generate a laser beam along the laser path;
metering power with a meter coupled to the laser;
gating the laser beam;
expanding the laser beam with a beam expander coupled to receive the laser beam to produce an expanded beam;
focusing the expanded beam onto the target region using a focusing lens located on the laser path;
positioning a hydrogel medium into a target region along a laser path wherein the hydrogel medium includes a monomer and at least one chromophore having a simultaneous two-photon absorptivity, wherein the chromophore is derived from a compound having the formula 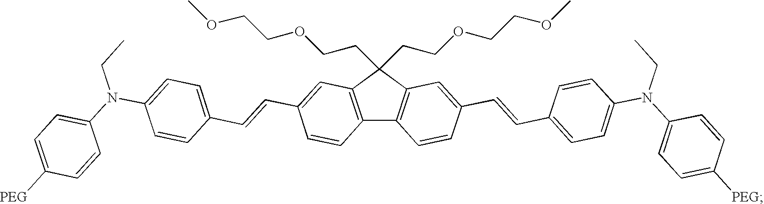
and
generating a structure by moving the sample in three dimensions relative to the focusing lens while irradiating said chromophore with the gated laser beam to cause a simultaneous two-photon absorption in said chromophore to produce two photon polymerization of the hydrogel medium.

12. An article, produced by the method of claim 11.

13. The method of claim 11 wherein the hydrogel medium comprises a compound having the formula 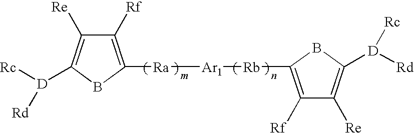
where:
D is selected from the group consisting of N, O, S;
m and n are independently selected from the range of integers greater than or equal to zero and less than or equal to six;
B is selected from group consisting CR1=CR2, O, S and N—R3 where R1, R2 and R3 are selected from the group consisting of;
(i) —H,
(ii) a linear or branched alkyl group with up to 10 carbons,
(iii) —(CH2CH2O)x-R, where x: (1-10), R is selected from the group consisting of H and linear branched alkyl group up to 10 carbons or an aryl group, and
(iv) an aryl group;
where Ra and Rb are selected from the group consisting of compositions having the following formulas: 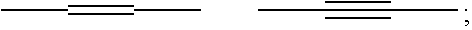
wherein at least one of Rc and Rd consists of a hydrogel structure;
wherein, one of Rc and Rd is not present when B is O or S;
wherein Ar₁ has a formulation selected from the group consisting of 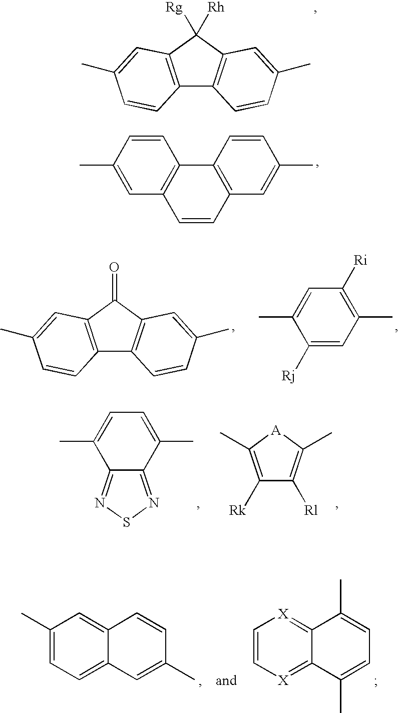
wherein Re, Rf, Rg, Rh, Ri, Rj, Rk, and Rl are selected from the group consisting of:
(i) —OR4, —CN, —SR5, —F, —Cl, —Br, —I, —NO2, —H,
(ii) a linear or branched alkyl group with up to 10 carbons,
(iii) —(CH2CH2O)x-R, where x: (1-10), R is selected from the group consisting of H and linear branched alkyl group up to 10 carbons,
(iv) an aryl group selected from the group consisting of phenyl, naphthyl, furanyl, thiophenyl, pyrrolyl, selenophenyl; and
wherein X is selected from the group consisting of C and N.

14. The method of claim 11 wherein the step of activating a laser comprises activating a femtosecond laser device.

15. The method of claim 14 wherein the femtosecond laser device comprises a fiber laser.

16. The method of claim 13 wherein the hydrolgel structure is selected from the group consisting of oligomer or polymer, collagen, collagen-GAG(alginate) copolymers, albumin, hyaluronic acid, fibrinogen-fibrin, chitosan, matrigel, alginate, polyhydroxyalkanoates, starch, poly(lactic acid), poly(d-lactic acid), poly(l-lactic acid), poly(d,l-lactic acid), poly(glycolic acid), poly(lactic-co-glycolic acid), poly(e-caprolactone), poly(hydroxyalkanoate), poly(3 or 4-hydroxybutyrate), poly(3-hydroxyoctanoate), poly(3-hydroxyvalerate), poly (p-dioxanone), poly(propylene fumarate), poly (1,3-trimethylene carbonate), poly(glycerol-sebacate), poly(ester urethane), polyethylene glycol, hydroxyethyl methacrylate (HEMA) and combinations thereof.

17. A compound or composition, comprising:
at least one chromophore initiator compatible with a hydrogel oligomer or polymer, wherein the chromophore has a simultaneous two-photon absorptivity; and
at least one monomer in close proximity to said chromophore, wherein the at least one monomer includes a hydrogel oligomer or polymer.

18. The compound or composition of claim 17 wherein the chromophore initiator comprises a constitutional unit derived from the formula 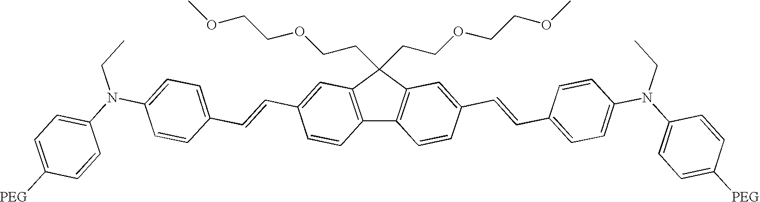
wherein PEG consists of polyethylene glycol.

19. The compound or composition of claim 17 wherein the at least one monomer comprises a compound having the formula 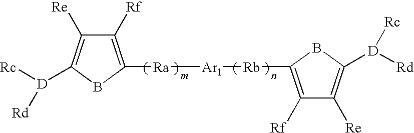
wherein D is selected from the group consisting of N, O, S;
wherein m and n are independently selected from the range of integers greater than or equal to zero and less than or equal to six;
wherein B is selected from group consisting CR1=CR2, O, S and N—R3, wherein R1, R2 and R3 are selected from the group consisting of
(v) —H,
(vi) A linear or branched alkyl group with up to 10 carbons,
(vii) —(CH2CH2O)x-R, where x: (1-10),
R is selected from the group consisting of H and linear branched alkyl group up to 10 carbons or an aryl group, and
(viii) An aryl group;
wherein Ra and Rb are selected from the group consisting of the following formulas: 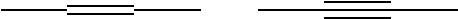
wherein at least one of Rc and Rd consists of a hydrogel oligomer or polymer;
wherein, one of Rc and Rd is not present when B is O or S;
wherein Ar₁ has a formulation selected from the group consisting of 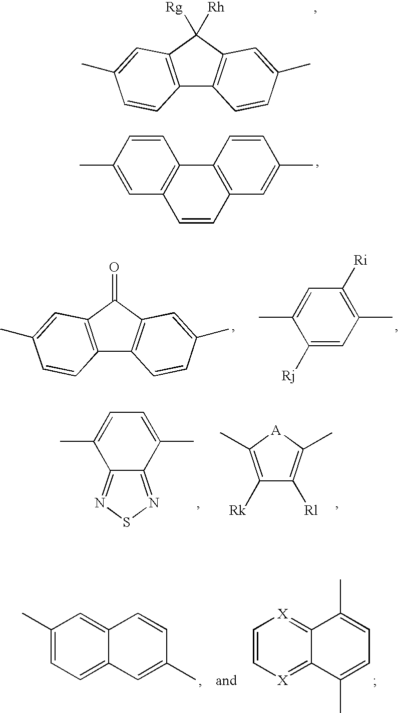
wherein Re, Rf, Rg, Rh, Ri, Rj, Rk, and Rl are selected from the group consisting of
(v) —OR4, —CN, —SR5, —F, —Cl, —Br, —I, —NO2, —H,
(vi) a linear or branched alkyl group with up to 10 carbon,
(vii) —(CH2CH2O)x-R, where x: (1-10), R is selected from the group consisting of H and linear branched alkyl group up to 10 carbons,
(viii) an aryl group selected from the group consisting of phenyl, naphthyl, furanyl, thiophenyl, pyrrolyl, selenophenyl; and
wherein X is selected from the group consisting of C and N.

20. The method of claim 17 wherein the hydrolgel oligomer or polymer is selected from the group consisting of collagen, collagen-GAG(alginate) copolymers, albumin, hyaluronic acid, fibrinogen-fibrin, chitosan, matrigel, alginate, polyhydroxyalkanoates, starch, poly(lactic acid), poly(d-lactic acid), poly(l-lactic acid), poly(d,l-lactic acid), poly(glycolic acid), poly(lactic-co-glycolic acid), poly(e-caprolactone), poly(hydroxyalkanoate), poly(3 or 4-hydroxybutyrate), poly(3-hydroxyoctanoate), poly(3-hydroxyvalerate), poly (p-dioxanone), poly(propylene fumarate), poly (1,3-trimethylene carbonate), poly(glycerol-sebacate), poly(ester urethane), polyethylene glycol, hydroxyethyl methacrylate (HEMA) and combinations thereof.

21. The compound or composition of claim 17 wherein the monomer structure is derived from the formula

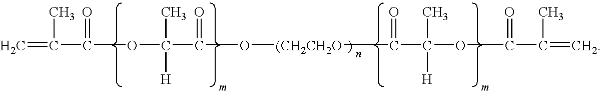

22. The compound or composition of claim 19 wherein the hydrolgel oligomer or polymer is selected from the group consisting of collagen, collagen-GAG(alginate) copolymers, albumin, hyaluronic acid, fibrinogen-fibrin, chitosan, matrigel, alginate, polyhydroxyalkanoates, starch, poly(lactic acid), poly(d-lactic acid), poly(l-lactic acid), poly(d,l-lactic acid), poly(glycolic acid), poly(lactic-co-glycolic acid), poly(e-caprolactone), poly(hydroxyalkanoate), poly(3 or 4-hydroxybutyrate), poly(3-hydroxyoctanoate), poly(3-hydroxyvalerate), poly (p-dioxanone), poly(propylene fumarate), poly (1,3-trimethylene carbonate), poly(glycerol-sebacate), poly(ester urethane), polyethylene glycol, hydroxyethyl methacrylate (HEMA) and combinations thereof.

23. The method of claim 19 wherein the monomer structure is derived from the formula [chemical structure]
